(19) United States
(12) Patent Application Publication
(10) Pub. No.: US 2006/0140990 A1
(43) Pub. Date: Jun. 29, 2006

(54) Composition for topical treatment of mixed vaginal infections

(75) Inventors: Jonathan Bortz, Saint Louis, MO (US); R. Saul Levinson, Chesterfield, MO (US); Mitchell Kirschner, St. Louis, MO (US); Robert C. Cuca, Glen Carbon, IL (US)

Correspondence Address: HARNESS, DICKEY, & PIERCE, P.L.C, 7700 BONHOMME, STE 400, ST. LOUIS, MO, 63105, US

(73) Assignee: DrugTech Corporation, Wilmington, DE (US)

(21) Appl. No.: 11/326,979

(22) Filed: Jan. 5, 2006

Related U.S. Application Data
(63) Continuation-in-part of application No. 10/944,416, filed on Sep. 20, 2004.
(60) Provisional application No. 60/507,138, filed on Oct. 1, 2003.
(60) Provisional application No. 60/504,017, filed on Sep. 19, 2003.

(51) Int. Cl. A61K 31/7034; A61K 9/00

(52) U.S. Cl. 424/400; 514/35

(57) ABSTRACT

A pharmaceutical composition comprises (a) an antibacterial agent in an antibacterially effective amount, illustratively comprising clindamycin or a pharmaceutically acceptable salt or ester thereof, and (b) an antifungal agent in an antifungally effective amount, illustratively comprising butoconazole or a pharmaceutically acceptable salt or ester thereof. The composition is adapted for application in a unit dose amount to a vulvovaginal surface and has at least one nonlipoidal internal phase and at least one lipoidal external phase that is bioadhesive to the vulvovaginal surface. The composition is useful for administration to a vulvovaginal surface to treat a mixed bacterial vaginosis and vulvovaginal candidiasis infection.

This application is a continuation-in-part of application Ser. No. 10/944,416, filed on Sep. 20, 2004, which claims priority of provisional application Ser. No. 60/507,138, filed on Oct. 1, 2003 and provisional application Ser. No. 60/504,017, filed on Sep. 19, 2003. Each of the above-cited applications is incorporated in its entirety herein by reference.

### FIELD OF THE INVENTION

The present invention relates to pharmaceutical compositions suitable for vaginal delivery of an antifungal agent and an antibacterial agent. The invention further relates to therapeutic methods of use of such compositions in women having mixed fungal and bacterial infections of the vulvovaginal system.

### BACKGROUND OF THE INVENTION

Infective vaginitis covers a range of conditions involving microbial infection of the vagina, and inflammation associated therewith, that sometimes extends to the vulva. It accounts for an estimated 15 million physician office visits a year in the U.S., and with availability of over-the-counter remedies particularly for candidal infections, many additional cases are medicated without professional diagnosis.

Agents of infection implicated in vaginitis include:

(a) fungi, more particularly yeasts, especially Candida spp. including one or more of C. albicans, C. dubliniensis, C. glabrata, C. kefyr, C. krusei, C. lusitaniae, C. neoformans, C. parasilopsis and C. tropicalis, of which the most common is C. albicans;

(b) bacteria, commonly a variety of species including one or more of Bacteroides spp., Gardnerella vaginalis, Mobiluncus spp., Mycoplasma hominis and Peptostreptococcus spp., most commonly with G. vaginalis predominating; and

(c) protozoa, especially Trichomonas vaginalis.

Candidal infections, herein referred to collectively as vulvovaginal candidiasis (VVC), are the best known cause of vaginitis and are believed to affect about 75% of women at least once during their lifetime. VVC is generally not sexually transmitted. Bacterial vaginosis (BV), a collective term used herein for vaginal or vulvovaginal conditions caused by bacterial infection, is generally considered a sexually transmitted disease although other modes of transmission can occur. Symptoms of VVC and BV include irritation (manifesting, for example, as redness, burning and/or itching), dyspareunia and abnormal discharge, which in the case of BV tends to have a fishy odor. Other diagnostic criteria include a vaginal pH lower than about 4.7 in VVC, or higher than about 4.7 in BV, and presence of “clue cells” (epithelial cells having a granular appearance) in BV.

VVC is typically a nuisance, often very troubling to the patient but relatively rarely implicated in development of more serious or life-threatening conditions. On the other hand, BV, if untreated, can lead to serious conditions, such as cervicitis, pelvic inflammatory disease, cervical dysplasia, urinary tract infections, postoperative infections, increased susceptibility to viral infection including HIV and HSV-2, and, in pregnant women, premature birth, preterm rupture of membranes, intra-amniotic fluid infection, preterm labor and postpartum endometritis.

Bacterial and candidal infections can coexist. Mixed bacterial and candidal (herein “BV/VVC”) infection occurs in up to about one-fifth of vaginitis cases. For example, Redondo-Lopez et al. (1990), Sex. Transm. Dis. 17(1):51-53, reported that in 132 episodes of symptomatic vaginitis in 35 patients with recurring symptoms, 15% were found to involve a mixed BV/VVC infection.

In another study, Ferris et al. (2002), Obstet. Gynecol. 99(3):419-425, reported that of 95 women who were about to treat themselves for VVC, 34% were confirmed to have VVC alone, 19% had BV alone, and 19% had a mixed BV/VVC infection.

A significant problem is that such mixed infections are underdiagnosed, and self-medication or prescribed treatment occurs as if for fungal or bacterial infection alone. Both fungi such as Candida albicans and bacteria such as Gardnerella vaginalis are opportunistic pathogens, therefore in case of a mixed infection removal of one can lead to rapid population growth of the other. Thus, for example, a mixed BV/VVC infection treated topically only with an antifungal agent such as butoconazole can quickly become a serious BV infection, which then requires follow-up antibacterial treatment, either as a further topical application or as systemic (e.g., oral antibiotic) therapy. Implications of such misdiagnosis can be nontrivial, especially considering the serious conditions to which BV can lead if untreated.

Thus a need exists in the art for a medicament and method of use thereof that conveniently and effectively treats mixed BV/VVC infections.

U.S. Pat. No. 4,551,148 to Riley et al. proposes a controlled release system for vaginal drug delivery, comprising unit cells having a nonlipoidal internal phase and a lipoidal continuous external phase. An active agent is present at least in the internal phase.

U.S. Pat. No. 5,266,329 to Riley proposes such a vaginal delivery system having an antifungal imidazole, exemplified by metronidazole, as the active agent.

Thompson & Levinson (2002), Drug Delivery Systems & Sciences 2(1), 17-19, describe a bioadhesive topical drug delivery system known therein as the VagiSite system as a high internal phase ratio water-in-oil emulsion system, providing a delivery platform for administration of active drug entities in the vaginal cavity. They disclose that the VagiSite system is incorporated in Gynazole-1® antifungal vaginal cream, which contains 2% by weight butoconazole nitrate.

U.S. Patent Application Publication No. 2003/0180366 of Kirschner et al. discloses a composition suitable for vaginal drug delivery, comprising an essentially pH neutral emulsion having an internal water-soluble phase and an external water-insoluble phase, wherein the internal phase comprises an acidic buffered phase comprising a drug, which can illustratively be an antifingal agent or an antibacterial agent. Example I therein provides such a composition comprising the antibacterial agent metronidazole in an amount of 0.75% by weight. Example II therein provides such a composition comprising the antibacterial agent clindamycin phosphate in an amount of 2.8% by weight.

U.S. Pat. No. 5,055,303 to Riley describes a solid composition, for example a suppository, comprising a water-in-oil emulsion that can carry an active agent. The composition is stated to be suitable for insertion into a body orifice and to melt at body temperature to form a cream having controlled release and bioadherent properties.

U.S. Patent Application Publication No. 2003/0225034 of Floros et al. mentions that, for treatment of vaginitis, surfactant lipids can be administered in conjunction with one or more medications including antibiotics and antifungals. Examples of antibiotics said to be suitable include ampicillin, ceftriaxone, clindamycin, metronidazole and tetracycline. Examples of antifungals said to be suitable include miconazole, clotrimazole, econazole, butoconazole, tioconazole and terconazole.

### SUMMARY OF THE INVENTION

There is now provided a pharmaceutical composition comprising (a) an antibacterial agent in an antibacterially effective amount; and (b) an antifungal agent in an antifungally effective amount. The composition is adapted for application to a vulvovaginal surface, for example a vaginal mucosal surface, and has at least one nonlipoidal internal phase and at least one lipoidal external phase that is bioadhesive to such a surface.

In one embodiment the antibacterial agent comprises clindamycin or a pharmaceutically acceptable salt or ester thereof, for example clindamycin phosphate; and the antifungal agent comprises butoconazole or a pharmaceutically acceptable salt or ester thereof, for example butoconazole nitrate.

The composition is typically a water-in-oil emulsion and can illustratively be presented in a semi-solid form described in the pharmaceutical art as a cream.

There is further provided a vaginal antibacterial and antifungal delivery system comprising such a cream and an applicator to facilitate administration to a vaginal mucosal surface.

There is still further provided a method for treating a mixed BV/VVC infection, the method comprising administering a pharmaceutical composition as described herein to a vulvovaginal surface, for example a vaginal mucosal surface.

In some embodiments, such a method can provide a “one dose to cure” treatment for the mixed infection.

These and other embodiments are more fully described in the detailed description that follows.

### DETAILED DESCRIPTION

The particular form of a composition useful herein is not limited and can be, for example, a cream, a gel, a foam, a vaginal tablet, pessary or suppository, a tampon, an implant such as a ring, etc.

However, of particular interest herein is a composition in the form of a water-in-oil emulsion as generally described in any of above-referenced U.S. Pat. No. 4,551,148, U.S. Pat. No. 5,055,303, U.S. Pat. No. 5,266,329 or U.S. Patent Application Publication No. 2003/0180366, or as further described herein. Such a water-in-oil emulsion can be presented in a solid form, for example as a vaginal suppository, or in a semi-solid form, for example as a vaginal cream, and has bioadhesive properties.

A “vulvovaginal surface” herein denotes any external or internal surface of the female genitalia, including mucosal surfaces in the vaginal cavity and nonmucosal surfaces of the vulva and immediately surrounding areas of skin. In some embodiments, the composition is more specifically adapted for application to a vaginal mucosal surface, and the external phase of the composition is bioadhesive, i.e., mucoadhesive, to such a surface.

In one embodiment, the composition is formulated as a bioadhesive vaginal delivery system as described by Thompson & Levinson (2002), op. cit. under the name VagiSite, or a vaginal delivery system substantially equivalent thereto, including as active agents an antibacterial agent and an antifungal agent.

International Patent Publication No. WO 2005/087270, incorporated herein by reference but not admitted to be prior art to the present invention, mentions the VagiSite system as an option for delivery of a combination of antivaginitis medicaments.

Bioadhesion, for example to a vaginal mucosal surface, is an important property of compositions of the invention. It is believed, without being bound by theory, that bioadhesion allows for a sustained and controlled delivery of the antibacterial agent, the antifungal agent, or both over time. Advantages over conventional vaginal delivery systems exhibiting less or no bioadhesion include one or more of:
(a) minimization of leakage of the composition from the site of application;
(b) suitability for application at any time of day, not limited to bedtime;
(c) reduction of active agent exposure, in particular systemic exposure, during a course of therapy;
(d) reduction of total active agent dose giving an acceptable clinical response;
(e) continuous active agent release during an extended period;
(f) more rapid relief of symptoms; and
(g) potential for single-dose therapy.

The bioadhesive property of a composition of the invention is believed, without being bound by theory, to reside at least in part in the lipoidal nature of the external phase of the composition, which repels moisture and thereby resists dilution and removal by normal vaginal secretion. It is further believed, again without being bound by theory, that the lipoidal external phase serves to sequester the internal nonlipoidal phase; in embodiments wherein the antibacterial agent, the antifungal agent or both are present partly or wholly in the internal phase, the active agent payload is likewise sequestered, allowing for release of the active agent to be metered slowly over time.

The bioadhesive and controlled or sustained release properties of a composition embodying a vaginal delivery system known as the Site Release® (SR) system useful herein have been demonstrated in studies summarized by Merabet et al. (2005), Expert Opin. Drug Deliv. 2(4):769-777, incorporated herein by reference but not admitted to be prior art to the present invention.

A “conventional” vaginal cream, used for example as a comparative composition in evaluating a vaginal cream composition embodying the SR system, herein refers to a semi-solid emulsion having a continuous aqueous or nonlipoidal phase and a discontinuous or disperse nonaqueous or lipoidal phase, i.e., an oil-in-water emulsion, wherein the active agent is solubilized or dispersed in the continuous phase. Typically, this permits immediate contact of the active agent with the vulvovaginal surface to which the composition is applied, but also permits dilution, rinsing and leakage of the composition from this surface, reducing the contact time with the surface and with the targeted bacterial and/or fungal pathogens. Conventional vaginal creams comprising an antibacterial agent and/or an antifungal agent therefore must generally be administered repeatedly, for example about 3 to 7 times a week, to provide a clinically acceptable response. Such repeated application increases the potential for systemic delivery of the active agent, and thereby increases the potential for adverse side-effects, and also increases likelihood of tissue irritation.

Weinstein et al. (1994), Clin. Ther. 16(6):930-934, studied the retention time of vaginal creams containing 2% butoconazole nitrate. A total of 16 healthy women were treated intravaginally with a conventional vaginal cream or a bioadhesive SR cream, and monitored daily over 7 days for the amount of residual cream detected within the vaginal cavity by gynecological swab. A median retention time of 4.2 days was reported for the SR cream, by comparison with about 2.5 days for the standard cream.

Thompson & Levinson (2002), op. cit., reported a study in which 28 healthy women received intravaginal treatment with a conventional antifungal vaginal cream or a bioadhesive SR cream containing the same antifungal agent, in either case as a single dose. The women wore mini-pads for a 48-hour period to evaluate product leakage from the vaginal cavity. At each time point studied (3, 6, 24 and 48 hours after administration), product leakage was reportedly greater with the conventional cream than with the SR cream. Overall, leakage was reduced by over 50% with the SR cream.

Conventional vaginal creams commonly require application at bedtime to take advantage of a supine position of the patient for several hours, which can help to retain the cream within the vaginal cavity. The bioadhesive property and consequently enhanced vaginal retention of a vaginal cream of the invention can enable application at any convenient time of day.

Thompson & Levinson (2002), op. cit., also reported in vitro analysis of butoconazole nitrate release properties of a conventional vaginal cream and a cream embodying the SR system, using a pH 4.3 acetate buffer, designed to simulate vaginal fluid. The conventional cream was reported to disintegrate rapidly and begin to release the active agent immediately, with substantially all of the active agent payload being released within 1 to 4 hours. By contrast, the SR cream was reported to release the active agent continuously over about 7 days.

The bioadhesive and sustained release properties of a vaginal cream of the invention can permit a relatively low dose of an active agent to provide a clinically acceptable response at least substantially equal to that provided by a much larger dose of the active agent administered in the form of a conventional cream. In particular, a single administration of a cream of the invention can provide a clinically acceptable response at least substantially equal to that provided by a conventional cream administered more than once, for example repeatedly about 3 to about 7 times in the course of one week. In this regard it is noted that adverse drug reactions are generally dose related, with appearance of new adverse events or exacerbation of existing adverse effects as the dose is escalated. A composition of the invention therefore has the potential to provide an improved safety profile. This is especially true with respect to adverse effects resulting from systemic delivery. The drug-sparing effect of a sustained release profile permitted by the present compositions tends to reduce systemic delivery yet still provides therapeutically effective delivery at the locus of administration.

A composition of the invention typically comprises a multiplicity of unit cells, which are the basic repeating units of the delivery system and are not divisible without losing at least some of the properties useful herein. Each unit cell has internal and external phases, corresponding to the internal and external phases of the composition referred to above. Compositions of the invention can be described using conventional classifications, for example as emulsions, emulsion/dispersions, double emulsions, suspensions within emulsions, suppositories, foams, creams, ovules, inserts, and so on. Usually compositions of the invention are in the form of water-in-oil emulsions having medium to high internal phase ratio (expressed as percentage of total volume occupied by the internal phase), for example greater than about 60%, greater than about 70%, or greater than about 75%, by volume.

Compositions of the invention include liquids or semi-solids having a viscosity of about 5,000 to about 1,000,000 centipoise, for example about 100,000 to about 800,000 centipoise. In certain embodiments the composition is a vaginal cream having a viscosity of about 5,000 to about 750,000 centipoise, for example about 350,000 to about 550,000 centipoise. A vaginal cream is generally a semi-solid water-in-oil emulsion and comprises an emulsifying agent. It is believed, without being bound by theory, that bioadherence of the composition to the vulvovaginal surface, for example the vaginal mucosal surface, requires that the composition have sufficient viscosity to retain its integrity when applied to such a surface. Optional ingredients that can increase viscosity, among other properties, include microcrystalline wax, colloidal silicon dioxide, and various pharmaceutically acceptable polymers including polysaccharides, cellulosic polymers such as carboxymethylcellulose, methylcellulose, hydroxypropylmethylcellulose, etc., polyethylene glycol, acrylate polymers and the like.

Solid compositions comprising a water-in-oil emulsion typically melt at body temperature to form a bioadhesive cream substantially as described above.

The internal phase is typically discontinuous and, as indicated above, is nonlipoidal. The nonlipoidal character of the internal phase renders it miscible with water. Illustratively, the internal phase comprises water, glycerin, propylene glycol, sorbitol or a combination of two or more thereof. Generally the internal phase has high osmotic pressure. The internal phase can itself be monophasic, biphasic or multiphasic, taking the form, for example, of a solution, suspension, emulsion or combination thereof. The internal phase optionally comprises one or more suspended solids, emulsifying and/or dispersing agents, osmotic enhancers, extenders, diluents, buffering agents, chelating agents, preservatives, fragrances, colors, or other materials.

Optionally, the internal phase is acid buffered to an internal pH of about 2.0 to about 6.0, for example about 2.5 to about 5.5 or about 3.5 to about 5.0. In one embodiment the internal phase is acid buffered to an internal pH that is substantially optimal to the vaginal environment, i.e., a pH that does not cause substantial irritation, itching or other discomfort and/or renders the vaginal environment less hospitable to common pathogens including fungal and bacterial pathogens. Typically such a pH is about 4.0 to about 5.0, for example approximately 4.5.

The external phase is typically continuous (in such systems adjacent unit cells have common external phases) and, as indicated above, is lipoidal. The term “lipoidal” herein can pertain to any of a group of organic compounds including neutral fats, fatty acids, waxes, phosphatides, petrolatum, fatty acid esters of monoprotic alcohols, mineral oils, etc., having the following properties: insoluble in water; soluble in alcohol, ether, chloroform or other fat solvents; and exhibiting a greasy feel. Examples of suitable oils are mineral oils having viscosity of about 5.6 to about 68.7 centistokes, for example about 25 to about 65 centistokes, and vegetable oils such as coconut, palm kernel, cocoa butter, cottonseed, peanut, olive, palm, sunflower, sesame, corn, safflower, rapeseed (canola) and soybean oils and fractionated liquid triglycerides of naturally derived short-chain fatty acids.

The term “lipoidal” can also pertain to amphiphilic compounds, including for example natural and synthetic phospholipids. Suitable phospholipids can include, for example phosphatidylcholine esters such as dioleoylphosphatidylcholine, dimyristoyl-phosphatidylcholine, dipentadecanoylphosphatidylcholine, dipalmitoylphosphatidyl-choline (DPPC) and distearoylphosphatidylcholine (DSPC); phosphatidylethanolamine esters such as dioleoylphosphatidylethanolamine and dipalmitoylphosphatidylethanol-amine (DPPE); phosphatidylserine; phosphatidylglycerol; phosphatidylinositol; etc.

In one embodiment, the external phase comprises a phospholipid component, for example a lecithin component, more particularly a refined lecithin component. Without being bound by theory, it is believed that refined lecithins or other phospholipid materials can reside at the oil-water interface of a water-in-oil emulsion and impart improved stability to the emulsion, especially where an active agent is present having surfactant properties that tend to disrupt emulsion stability. A preferred lecithin comprises not less than about 70%, for example not less than about 80%, phosphatidylcholine. The phosphatidylcholine content of the lecithin can be as high as about 96% or even higher. Food grade lecithin may or may not be found acceptable in specific formulations. An example of a refined lecithin that is generally suitable is Phospholipon 90™, available from American Lecithin Co.

Amphiphilic compounds other than phospholipids can also act, optionally together with a phospholipid, as emulsifying agents in a composition of the invention. Any pharmaceutically acceptable emulsifying agent or combination thereof can be used, including without limitation medium and long chain monoglycerides and diglycerides, such as glyceryl monooleate, glyceryl monostearate, glyceryl monoisostearate and glyceryl monopalmitate, polyglyceryl esters of fatty acids, such as polyglyceryl-3 oleate, and polyethylene glycol esters and diesters of fatty acids, such as PEG-30 dipolyhydroxystearate. Such agents can also function as emollients in the composition. Emulsifying agents soluble in the external phase are generally preferred. In one embodiment a mono- and diglyceride mixture is used, alone or with addition of a metallic soap such as aluminum stearate.

Water-in-oil emulsion compositions of the invention are typically deformable at physiological temperatures (approximately 37° C.) but, unlike conventional creams, do not rapidly lose integrity upon application to a vaginal mucosal surface. In general, therefore, they do not result in offensive or otherwise unacceptable leakage from the vaginal cavity following administration. As physical breakdown of such compositions occurs over an extended period, nonaqueous components are either absorbed or released from the vaginal cavity at a generally unnoticeable rate, making no substantial increase over normal rates of vaginal secretion.

Release of the antibacterial agent, the antifungal agent or both from a composition of the invention can occur by one or more mechanisms, none of which are limiting to the present invention. Such mechanisms can include diffusion, for example from the internal phase through the external phase into the vaginal mucosa; rupture of unit cells; dissolution of solid particulates; etc. Release dynamics can be linear or nonlinear.

Compositional factors affecting release rate of each active agent can include relative amounts of the active agent present in the internal and external phases; internal phase ratio; osmotic pressure of the internal phase; pH of the internal phase; selection and relative amounts of lipoidal compounds, including amphiphilic compounds, in the external phase, influencing diffusibility of the active agent therein; particle size where the active agent is in solid particulate form; viscosity of the composition; etc. Each of these factors can be routinely modified by one of skill in the art based on the disclosure herein, to optimize release rate for specific situations. In a composition having the active agent in the internal phase, and having a relatively small internal phase ratio, the external phase tends to form a relatively thick membrane through which the active agent must pass to be released; accordingly release rate can be significantly slowed in such a composition.

Physiological factors affecting release rate of each active agent include factors affecting rate of physical breakdown or loss of integrity of the composition, such as amount and chemical nature of fluids and enzymes, pH, chemical balance, temperature and shear forces arising from body movement. Shear forces are believed not to affect integrity of compositions of the invention as rapidly or severely as in the case of conventional vaginal creams.

The composition is typically adapted to release the antibacterial agent, the antifungal agent or both over a period of about 3 hours to about 10 days, upon application to a vulvovaginal surface, for example a vaginal mucosal surface. Based on the disclosure herein, including disclosure of documents incorporated by reference herein, in particular above-referenced U.S. Pat. Nos. 4,551,148 and 5,266,329, U.S. Patent Application Publication No. 2003/0180366 and the parent application of this continuation-in-part application (published as U.S. Patent Application Publication No. 2005/0095245 but not admitted herein to be prior art to the present invention), one of skill in the art can without undue experimentation adjust release rate of each active agent from the composition to achieve a release period of about 3 hours to about 10 days. In various embodiments, the release period is one of about 12 hours to about 10 days, about 1 to about 10 days, about 2 to about 10 days or about 3 to about 7 days.

A wide range of release profiles is thus possible for each active agent. In one embodiment, at least one of the active agents exhibits, by 1 day after administration, about 2% to about 25% release; by 2 days after administration, about 15% to about 50% release; by 3 days after administration, about 25% to about 75% release; and by 4 days after administration, about 45% to 100% release.

Release rate can be determined by in vivo testing or by any suitable in vitro method. An illustrative in vitro method utilizes an open chamber diffusion cell system such as a Franz cell system, typically fitted with an appropriate inert synthetic membrane such as polysulfone, cellulose acetate/nitrate mixed ester or polytetrafluoroethylene of suitable thickness, e.g., 70 μm. The receptor medium should be one in which the active agent of interest is soluble, for example a water/ethanol medium. A test composition is placed uniformly on the membrane (illustratively, about 300 mg of a semi-solid composition such as a cream is a suitable amount for placement on a 25 mm diameter membrane) and is kept occluded to prevent solvent evaporation and compositional changes. This corresponds to an infinite dose condition. An aliquot of the receptor fluid is removed for analysis at appropriate intervals, and is replaced with an aliquot of fresh receptor fluid, so that the membrane remains in contact with the receptor fluid throughout the period of the release study. A release rate study such as that outlined above is typically replicated and can be conducted using a standard composition having known release properties for comparison.

A “release period” or equivalent phrase herein refers to a period during which the active agent is made available for absorption and pharmacological (in the present case antibacterial or antifungal) effect, such effect typically occurring at or close to the site of absorption, for example the vaginal cavity. Thus the “release period” begins when release substantially begins (e.g., immediately to about 1 hour after administration, or later in the case of a delayed-release composition), and ends when substantially no further active agent is available for release (e.g., about 3 hours to about 10 days after the beginning of the release period).

The antibacterial agent, the antifungal agent or both can be present in either one or both of the internal and external phases. In one embodiment both agents are present at least in part in the internal phase of the composition, and can be in dispersed form, for example in solution or suspension therein, or in non-dispersed form. Optionally, substantially all of the antibacterial agent and/or substantially all of the antifungal agent can be present in the internal phase. Solubilization of one or both agents can be achieved, for example, by use of a cosolvent and/or surfactant. Some agents, for example the antibacterial agents metronidazole and clindamycin phosphate, are fairly water soluble or readily solubilized, and such agents are typically present at least in part in solution in the internal phase. Commonly, however, one or both agents can be present at least in part in particulate form, for example in micronized form or in nanoparticulate form, and can be dispersed as a particulate suspension in the internal and/or external phase. In various embodiments the antibacterial agent, the antifungal agent or both are present in aggregates or liposomes within the internal and/or external phase.

In compositions having one or both active agents in solid particulate form, any suitable particle size can be used. Typically, however, good physical stability may be difficult to achieve where a substantial portion of the particles are greater than about 250 μm in diameter. Thus a D₉₀ particle size (wherein 90% by weight of the particles are smaller than the specified size) not greater than about 250 μm is generally desirable for both the antibacterial agent and the antifungal agent. Preferably at least 99% by weight of the particles are not greater than about 250 μm in diameter.

Particle sizes smaller than about 5 μm can be useful but the expense of particle size reduction may not be justified by any improvement in stability or efficacy at such particle sizes. Nonetheless, particle sizes as small as 0.4 μm (400 nm), or even as small as 50 nm, can be used if desired.

The antibacterial agent can comprise any antibacterial known in the art to be useful in treatment of bacterial infections of the vulvovaginal system. The antibacterial can be one predominantly targeting a particular category of pathogenic bacteria, for example aerobic, anaerobic, gram-negative, gram-positive, etc. Illustrative examples of antibacterials that can be useful include without limitation acriflavine, ampicillin, ceftriaxone, chloramphenicol, chlorquinaldol, clindamycin, iodoquinol, metronidazole, nimorazole, omidazole, pivampicillin, secnidazole, spiramycin, tetracycline, tinidazole, pharmaceutically acceptable salts and esters thereof, mixtures thereof and the like. In one embodiment the antibacterial agent comprises or consists essentially of clindamycin or a pharmaceutically acceptable salt or ester thereof, for example clindamycin hydrochloride or clindamycin phosphate. In a particular embodiment the antibacterial agent comprises or consists essentially of clindamycin phosphate. The antibacterial agent is present in the composition in an antibacterially effective amount.

Amounts of clindamycin or a salt or ester thereof are expressed herein as clindamycin (free base) equivalent amounts unless the context demands otherwise. Any antibacterially effective amount of clindamycin or salt or ester thereof can be used, but typically in a vaginal cream preparation a clindamycin equivalent amount of about 0.5% to about 6% by weight, for example about 1% to about 3% by weight, will be found useful.

The antifungal agent can comprise any antifungal known in the art to be useful in treatment of fungal, especially candidal, infections of the vulvovaginal system. Illustrative antifungal agents include without limitation atovaquone, griseofulvin, nystatin, polymyxin B, terbinafine, and imidazole and triazole compounds such as butoconazole, clotrimazole, econazole, fluconazole, isoconazole, itraconazole, ketoconazole, miconazole, oxiconazole, ravuconazole, saperconazole, sertaconazole, sulconazole, terconazole, tioconazole, voriconazole, pharmaceutically acceptable salts and esters thereof, mixtures thereof and the like. In one embodiment the antifungal agent comprises or consists essentially of butoconazole or a pharmaceutically acceptable salt or ester thereof. In a particular embodiment the antifungal agent comprises or consists essentially of butoconazole nitrate. The antifungal agent is present in the composition in an antifungally effective amount.

Amounts of butoconazole or a salt or ester thereof are expressed herein as butoconazole nitrate equivalent amounts unless the context demands otherwise. Any antifungally effective amount of butoconazole or salt or ester thereof can be used, but typically in a vaginal cream preparation a butoconazole nitrate equivalent amount of about 0.5% to about 6% by weight, for example about 1% to about 3% by weight, will be found useful.

It will be recognized by one of skill in the art that the terms “antibacterial” or “antifungal”, applied to an active agent herein, are not necessarily mutually exclusive. A particular agent can exhibit, to some degree, both antifungal and antibacterial activity. Some agents, for example certain imidazoles including metronidazole, are utilized herein principally for their antibacterial activity, but also possess a useful degree of antifungal (including anticandidal), and in some cases antiprotozoal (including antitrichomonal) activity. Where such an agent is included in a composition of the invention as an antibacterial agent, some additional benefit is therefore possible in supplementing the activity of the antifungal agent (e.g., butoconazole) against a fungal pathogen such as C. albicans.

In one embodiment, one active agent, for example the antibacterial metronidazole, is present at least in substantial part in the internal phase of the composition and is substantially solubilized therein, and another active agent, for example the antifungal butoconazole nitrate, is likewise present at least in substantial part in the internal phase but is substantially in particulate form and suspended therein.

A particular example of a vaginal cream composition of the invention comprises clindamycin phosphate in a clindamycin equivalent amount of about 2% by weight, and butoconazole nitrate in an amount of about 2% by weight. The composition has (i) at least one nonlipoidal internal phase, (ii) at least one lipoidal external phase that is bioadhesive to a vaginal mucosal surface; and (iii) an emulsifying agent, for example comprising a phospholipid. The clindamycin phosphate and butoconazole nitrate are present at least in substantial part in the internal phase.

Another particular example of a vaginal cream composition of the invention comprises metronidazole in an amount of about 0.75% by weight, and butoconazole nitrate in an amount of about 2% by weight. The composition has (i) at least one nonlipoidal internal phase, (ii) at least one lipoidal external phase that is bioadhesive to a vaginal mucosal surface, and (iii) an emulsifying agent, for example comprising a phospholipid. The metronidazole and butoconazole nitrate are present at least in substantial part in the internal phase.

Illustratively, excipient ingredients in a vaginal cream composition of the invention can include water, sorbitol (e.g., in the form of a sorbitol solution), lecithin, at least one long chain monoglyceride, for example glyceryl monooleate, glyceryl monostearate, glyceryl monoisostearate or glyceryl monopalmitate, at least one polyglyceryl or polyethylene glycol fatty acid ester, for example polyglyceryl-3 oleate or PEG-30 dipolyhydroxystearate, a chelating agent, for example edetate disodium, at least one antimicrobial preservative, for example methylparaben and/or propylparaben, mineral oil and microcrystalline wax.

A unit dosage amount of a composition of the invention is an amount suitable for a single administration to a vulvovaginal surface, for example a vaginal mucosal surface, as described herein. Most conveniently for the patient, the composition is provided in unit dose aliquots, typically individually packaged, but this is not a requirement of the present invention. A convenient unit dose aliquot of a vaginal cream is an amount of about 1 to about 10 g, although greater or lesser amounts, for example as little as about 0.1 g or as much as about 25 g, can be used if desired. A particularly suitable unit dosage amount of a vaginal cream is about 3 to about 6 g, for example about 5 g. Where a unit dosage amount is smaller, it may be desirable to increase the active agent concentration in the composition, and vice versa.

Conveniently, a unit dosage amount of a vaginal cream of the invention can be furnished in a prefilled container or applicator, for example an applicator similar to that used for Gynazole-1® vaginal cream of KV Pharmaceutical Co., St Louis, Mo.

An antibacterial and antifungal delivery system comprising a vaginal cream composition of the invention, for example a disposable applicator, more particularly a disposable applicator prefilled with a unit dosage amount of the composition, is an embodiment of the invention.

A composition of the invention in the form of a vaginal cream can be prepared by known batch or continuous processes for preparing pharmaceutical creams. As in preparing conventional emulsions, shear force is applied to the components by use of a mixer, homogenizer, mill, impingement surface, ultrasound, shaking or vibration. However, unlike conventional emulsions, water-in-oil emulsions of the invention should normally be prepared using mixing shear at a relatively low level to prevent destruction of the emulsion by excess energy.

Illustratively, the internal and external phases are first prepared separately. In a typical batch process, the internal phase is added to the external phase while mixing in a planetary-type or other suitable mixer until a stable emulsion is formed. Addition rates and mixing speeds can be adjusted to optimize formation and viscosity of the emulsion. In a typical continuous process, the external phase is introduced into a continuous mixer that comprises a plurality of impellers, until it reaches the level of the lowest impeller in the mixing chamber. The two phases are then simultaneously introduced through the bottom of the mixer in proper proportion as the impellers rotate to apply shear to the components. The finished emulsion emerges through the top of the mixer. Flow rate through the mixing chamber and mixing speed can be adjusted to optimize formation and viscosity of the emulsion.

A composition of the invention can be administered topically to external surfaces of the vulva and/or to surrounding areas of skin. In addition or alternatively, the composition can be administered intravaginally. In one embodiment, the composition is a vaginal cream and is administered intravaginally in a unit dosage amount as defined above to a vaginal mucosal surface.

A vaginal cream of the invention can be administered to contact a mucosal surface in the vaginal cavity by means, for example, of an applicator that is optionally pre-filled with a single unit dosage amount of the cream. With the patient in a supine position, the tip of the applicator can be gently inserted high in the vagina, for example in the posterior vaginal formix, and the cream can be released through the tip by pushing on a plunger of the applicator.

A method of the invention for treating a mixed BV/VVC infection comprises administering a pharmaceutical composition, for example a vaginal cream composition, as described herein to a vulvovaginal surface, for example a vaginal mucosal surface. Such a method can also be used for treating a secondary condition arising from such a mixed infection.

Such a method can involve repeated administration of a unit dosage amount of the composition until a clinically acceptable response is obtained; however, it is an advantage of at least some compositions of the invention over conventional vaginal creams that a clinically acceptable response is often obtainable with a single administration. A method wherein a single administration of a unit dosage amount provides a clinically acceptable response is often known as a “one dose to cure” therapy, but it will be recognized that the term “cure” in the present context does not necessarily mean total or permanent removal of the infection or total or permanent relief from all symptoms.

A clinically acceptable response or “cure” herein can be illustratively evidenced by one or more of the following outcomes:
(a) resolution of all four clinical “Amsel criteria”, namely normal vaginal discharge, vaginal pH <4.7, <20% clue cells on wet mount, and negative “whiff” test, as described by Amsel et al. (1983), Am. J. Med. 74:14-22;
(b) a “Nugent score”<4 by the gram stain interpretation method of Nugent et al. (1991), J. Clin. Microbiol. 29:297-301; and
(c) a physician's negative answer to the question, “In your opinion, does the patient require additional treatment for BV/VVC at this time?”

In one embodiment, a therapeutic method using a composition of the invention provides, by a single administration, a “cure” rate at least substantially equal to that provided by about 3 to about 7 applications of a conventional vaginal cream composition, containing the same antibacterial and antifungal agents at the same concentration as the composition of the invention, in the course of one week.

A method of the invention can be used for treatment of any combination of bacterial and fungal infections present in the vulvovaginal system, including without limitation infections involving:
(a) fungi, more particularly yeasts, especially Candida spp. including one or more of C. albicans, C. dubliniensis, C. glabrata, C. kefyr, C. krusei, C. lusitaniae, C. neoformans, C. parasilopsis and C. tropicalis, of which the most common is C. albicans; and
(b) bacteria, commonly a variety of species including one or more of Bacteroides spp., Gardnerella vaginalis, Mobiluncus spp., Mycoplasma hominis and Peptostreptococcus spp., most commonly with G. vaginalis predominating.

A further list of bacterial species identified in women with BV has been reported by Fredricks et al. (2005), N. Engl. J. Med. 353:1899-1911, incorporated herein by reference but not admitted to be prior art to the present invention.

### EXAMPLES

The following examples are merely illustrative, and do not limit this disclosure in any way.

The vaginal cream compositions detailed below can be prepared by any method known in the art for preparing semi-solid emulsions, including batch and continuous processes as described hereinabove.

### Example 1

### Vaginal Cream Clindamycin+Butoconazole

| Ingredient | % w/w |
|---|---|
| water, purified, USP | 41.32 |
| sorbitol solution, USP | 37.20 |
| edetate disodium, USP | 0.05 |
| clindamycin phosphate, USP | 2.80 * |
| butoconazole nitrate, USP | 2.00 |
| mineral oil, USP | 10.00 |
| PEG-30 dipolyhydroxystearate | 4.00 |
| glyceryl monoisostearate | 2.00 |
| microcrystalline wax, NF | 0.40 |
| methylparaben, NF | 0.18 |
| propylparaben, NF | 0.05 |
| Total | 100.00 |

* equivalent to 2.00% clindamycin

### Example 2

### Vaginal Cream Metronidazole+Butoconazole

| Ingredient | % w/w |
|---|---|
| water, purified, USP | 41.32 |
| sorbitol solution, USP | 37.20 |
| edetate disodium, USP | 0.05 |
| metronidazole, USP | 0.75 |
| butoconazole nitrate, USP | 2.00 |
| mineral oil, USP | 10.00 |
| PEG-30 dipolyhydroxystearate | 4.00 |
| glyceryl monoisostearate | 2.00 |
| microcrystalline wax, NF | 0.40 |
| methylparaben, NF | 0.18 |
| propylparaben, NF | 0.05 |
| Total | 100.00 |

All patents and publications cited herein are incorporated by reference into this application in their entirety.

The words “comprise”, “comprises”, and “comprising” are to be interpreted inclusively rather than exclusively.

What is claimed is:

1. A pharmaceutical composition comprising:
(a) an antibacterial agent in an antibacterially effective amount; and
(b) an antifungal agent in an antifungally effective amount;
the composition being adapted for application to a vulvovaginal surface and having at least one nonlipoidal internal phase and at least one lipoidal external phase that is bioadhesive to the vulvovaginal surface.

2. The composition of claim 1, wherein the antibacterial agent comprises clindamycin or a pharmaceutically acceptable salt or ester thereof.

3. The composition of claim 1, wherein the antifungal agent comprises butoconazole or a pharmaceutically acceptable salt or ester thereof.

4. The composition of claim 1, wherein the vulvovaginal surface to which the composition is adapted for application is a vaginal mucosal surface.

5. The composition of claim 4, wherein upon application of the composition to the vaginal mucosal surface the antibacterial and antifungal agents each have a release period of about 3 hours to about 10 days.

6. The composition of claim 5, wherein upon application of the composition to the vaginal mucosal surface the antibacterial and antifungal agents each have a release period of about 12 hours to about 10 days.

7. The composition of claim 5, wherein at least one of said agents exhibits, by 1 day after administration, about 2% to about 25% release; by 2 days after administration, about 15% to about 50% release; by 3 days after administration, about 25% to about 75% release; and by 4 days after administration, about 45% to 100% release.

8. The composition of claim 4 that is in a form of a vaginal cream.

9. The composition of claim 8, wherein the antibacterial agent comprises clindamycin or a pharmaceutically acceptable salt or ester thereof, and the antifungal agent comprises butoconazole or a pharmaceutically acceptable salt or ester thereof.

10. The composition of claim 9, wherein the clindamycin or salt or ester thereof is present in a clindamycin equivalent amount of about 0.5% to about 6% by weight.

11. The composition of claim 9, wherein the clindamycin or salt or ester thereof is present in a clindamycin equivalent amount of about 1% to about 3% by weight.

12. The composition of claim 9, wherein the butoconazole or salt or ester thereof is present in a butoconazole nitrate equivalent amount of about 0.5% to about 6% by weight.

13. The composition of claim 9, wherein the butoconazole or salt or ester thereof is present in a butoconazole nitrate equivalent amount of about 1% to about 3% by weight.

14. The composition of claim 8, wherein the antibacterial agent comprises clindamycin phosphate and the antifungal agent comprises butoconazole nitrate.

15. The composition of claim 1, wherein the internal phase is acid buffered to an internal pH of about 2.0 to about 6.0.

16. The composition of claim 1, wherein the internal phase is acid buffered to an internal pH that is substantially optimal to the vaginal environment.

17. The composition of claim 1, said composition being in a form of a vaginal cream comprising clindamycin phosphate in a clindamycin equivalent amount of about 2% by weight, and butoconazole nitrate in an amount of about 2% by weight; the composition having (i) at least one nonlipoidal internal phase; (ii) at least one lipoidal external phase that is bioadhesive to a vaginal mucosal surface; and (iii) an emulsifying agent; wherein the clindamycin phosphate and butoconazole nitrate are present at least in substantial part in the internal phase.

18. The composition of claim 17, wherein the emulsifying agent comprises a phospholipid.

19. A vaginal antibacterial and antifungal delivery system comprising the composition of claim 8 and an applicator.

20. The delivery system of claim 21, wherein the applicator is disposable.

21. The delivery system of claim 21, wherein the applicator is prefilled with a unit dose amount of the composition.

22. The delivery system of claim 23, wherein the unit dose amount of the composition is about 1 to about 10 g.

23. The delivery system of claim 23, wherein the unit dose amount of the composition is about 3 to about 6 g.

24. A method for treating a mixed bacterial vaginosis and vulvovaginal candidiasis infection, the method comprising administering to a vulvovaginal surface a pharmaceutical composition comprising:
(a) an antibacterial agent in an antibacterially effective amount; and
(b) an antifungal agent in an antifungally effective amount;
wherein the composition has at least one nonlipoidal internal phase and at least one lipoidal external phase that is bioadhesive to the vulvovaginal surface.

25. The method of claim 26, wherein:
(a) the antibacterial agent comprises clindamycin or a pharmaceutically acceptable salt or ester thereof in a clindamycin equivalent amount of about 0.5% to about 6% by weight; and
(b) the antifungal agent comprises butoconazole or a pharmaceutically acceptable salt or ester thereof in a butoconazole nitrate equivalent amount of about 0.5% to about 6% by weight.

26. The method of claim 26, wherein the vulvovaginal surface to which the composition is administered is a vaginal mucosal surface.

27. The method of claim 28 wherein the composition is applied in a single dosage amount effective to provide an acceptable clinical response.

28. The method of claim 29, wherein the single dosage amount is about 1 to about 10 g.

29. The method of claim 26, comprising administering to a vaginal mucosal surface a single dosage amount of about 5 g of a vaginal cream composition comprising clindamycin phosphate in a clindamycin equivalent amount of about 2% by weight, and butoconazole nitrate in an amount of about 2% by weight; the vaginal cream composition having (i) at least one nonlipoidal internal phase; (ii) at least one lipoidal external phase that is bioadhesive to the vaginal mucosal surface; and (iii) an emulsifying agent; wherein the clindamycin phosphate and butoconazole nitrate are present at least in substantial part in the internal phase.
